(19) United States
(12) Patent Application Publication
(10) Pub. No.: US 2007/0056852 A1
(43) Pub. Date: Mar. 15, 2007

(54) Enzyme electrode sensor fuel cell and electrochemical reactor

(75) Inventors: Wataru Kubo, Kanagawa-ken (JP); Tetsuya Yano, Kanagawa-ken (JP); Tsuyoshi Nomoto, Tokyo (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 10/573,179

(22) PCT Filed: Jul. 22, 2005
(86) PCT No.: PCT/JP05/13907

§ 371 Date: Mar. 23, 2006

(30) Foreign Application Priority Data
Jul. 23, 2004 (JP) 2004-216377

(51) Int. Cl. C12Q 1/00; H01M 4/90

(52) U.S. Cl. 204/403.14; 429/43

(57) ABSTRACT

An enzyme electrode comprises a conductive member and an enzyme, wherein a first mediator and a second mediator are immobilized by a carrier onto the conductive member, the first mediator and the second mediator having different redox potentials (reduction-oxidation potentials). A sensor employs the enzyme electrode as a detection portion for detecting a substance. A fuel cell employs the enzyme electrode as at least one of anode and a cathode. An electrochemical reactor employs the enzyme electrode as a reaction electrode.

### TECHNICAL FIELD

The present invention relates to an enzyme electrode: more specifically, to an enzyme electrode comprising a conductive member; and a carrier, an enzyme, and a mediator immobilized on the conductive member. The present invention relates also to a sensor, and fuel cell employing the enzyme electrode; and a process for producing the enzyme electrode.

### BACKGROUND ART

An enzyme, a proteinaceous biocatalyst formed in a living cell, is highly active under mild conditions in comparison with ordinary catalysts. Further, the enzyme is highly specific to a substrate undergoing an enzymatic reaction, and catalyzes a specific reaction of a specific substrate. Ideally, the enzyme having such properties will enable preparation of a highly selective electrode having a low overvoltage, for an oxidation-reduction reaction on the electrode. However, the active centers of most redox enzymes (oxidoreductases) are usually enclosed in a deep interior of a three-dimensional structure of a glycoprotein, so that direct high-speed electron transfer cannot be caused between the oxidoreductase and the electrode. To promote the electron transfer, a method is disclosed which employs a substance called a mediator to connect electronically the enzyme with the electrode. In the early stage of the development, the enzyme and the mediator are dissolved in an electrolyte solution for simplicity of the experiment system and for freedom of motion of the enzyme and the mediator. Later, methods of immobilization thereof on the electrode are disclosed for improvement of the efficiency, prevention of leakage from the electrode, and continuous and long-term use of the electrode. The methods for immobilizing the enzyme and the mediator on the conductive member include adsorption, entrapping, crosslinking, and covalent bonding. Of these methods, entrapping, crosslinking, and covalent bonding give high ability for retaining the enzyme and the mediator and preventing leakage thereof from the system, enabling repeated use of the enzyme electrode in comparison with adsorption methods. Of these methods, in particular, entrapping methods are more advantageous owing to high retention capacity of the enzyme the mediator.

An important index of the performance of the enzyme electrode is an electric current density, which is an electric current intensity for a projected area of a conductive member. The higher current density enables, for example, improvement in detection sensitivity, simplification of a measurement portion, and miniaturization of a sensing part when used in a sensor based on current intensity detection; improvement of output when used as an electrode of a fuel cell; and improvement of reaction velocity when used as an electrochemical reactor, advantageously. The current density of the enzyme electrode can be increased by any of the methods of (1) increase of a turnover number (a number of substrate molecules converted by an enzyme in a unit time), (2) improvement of the electron transfer rate and efficiency between the enzyme and the mediator, (3) improvement of electron transportation in the mediator, (4) improvement of electron transfer rate and efficiency between the mediator and the conductive member, (5), increase of the enzyme-holding density (the amount of the enzyme per projected area of the conductive member), and so forth.

For the increase of the enzyme-holding density of the above item (5), immobilization methods which enable high enzyme-retention capacity are advantageous. Therefore, the above-mentioned entrapping methods are preferred which entrap the enzyme and the mediator in a carrier and immobilize the carrier on the surface of the conductive member. FIG. 1 illustrates schematically a state of immobilization of an enzyme by an entrapping method. In FIG. 1, enzyme 1 and mediator 2 are entrapped in carrier 3 fixed on a conductive member 4 (conducting substrate).

In the entrapping method, an electric charge produced on an enzyme by an enzyme/substrate reaction transfers from oxidation-reduction center of the enzyme to a mediator in a carrier, and is transported by electron hopping between the mediator to vicinity of a conductive member, and is finally detected by an external circuit by transfer of the charge between the mediator and the conductive member. (In FIG. 1, the flow of the charge is shown by arrow marks.) Therefore, even when the enzyme is immobilized in an amount larger than the enzyme full-occupation area for the effective surface area of the conductive member, the enzyme immobilization density for the projected area of the conductive member can be improved. For example, an enzyme electrode utilizing the entrapping immobilization for a fuel cell is disclosed in U.S. Pat. No. 6,531,239 (Heller et al.) in which the enzyme electrode is prepared by immobilization of an enzyme by a polymer containing a mediator in the molecule.

Generally, electron transfer velocity between an enzyme and a mediator is known to decrease exponentially with increase of the electron transfer distance between the oxidation-reduction center of the enzyme and the mediator. In the entrapping immobilization, since the enzyme and the mediator are not arranged in suitable distances or suitable orientation, the electron transfer between the enzyme and the mediator is considered not to occur before the enzyme and the mediator come close within an electron-transferable distance by random molecular motion. Accordingly, the electron transfer velocity between the enzyme and the mediator is not sufficient owing to dependence on the molecular motion of the both, and can be a limiting factor of the electric current density of the enzyme electrode.

On the other hand, in an example of the covalent bonding method, a mediator is bonded to a conducting substrate; the oxidation-reduction center of an enzyme is bonded to the mediator; and an enzyme excluding the oxidation-reduction center is bonded to the oxidation-reduction center bonding to the mediator. FIG. 2 illustrates immobilization state of an enzyme by a covalent bonding method. In this example of FIG. 2 mediator 6 and enzyme 1 are immobilized through a carrier substance onto the surface of conductive member 4 (conducting substrate). In FIG. 2, the flow of charges is shown by arrow marks.

Kats et al. (J. Am. Chem. Soc. 1996, vol. 118, pp. 10321-10322) discloses an enzyme electrode in which pyrroloquinolinequinone is bonded to flavin adenine dinucleotide as a mediator, and apoglucose oxidase having no oxidation-reduction center is reconstructed thereto. In this enzyme electrode, the mediator can be arranged near to the oxidation-reduction center of the enzyme in a distance, enabling rapid electron transfer with orientation. In this method, however, the mediator-enzyme pairs are bonded in a monomolecular state onto the conductive member, and the enzyme molecules cannot be immobilized in two or more layers, which limits the enzyme immobilization density and the increase of electric current density in the enzyme electrode.

### DISCLOSURE OF THE INVENTION

The present invention provides an enzyme electrode which is improved in electric current density. The present invention provides also a sensor, an electrochemical reactor, and a fuel cell utilizing the enzyme electrode.

According to an aspect of the present invention, there is provided an enzyme electrode comprising a conductive member and an enzyme, wherein a first mediator and a second mediator are immobilized by a carrier onto the conductive member, the first mediator and the second mediator having different redox potentials (reduction-oxidation potentials).

The first mediator preferably has a redox potential (reduction-oxidation potential) more negative than the redox potential (reduction-oxidation potential) of the second mediator, and the electron transfer reaction rate between the second mediator and the conductive member is higher than the electron transfer reaction rate between the first mediator and the conductive member.

The first mediator preferably has an redox potential (reduction-oxidation potential) more positive than the redox potential (reduction-oxidation potential) of the second mediator, and the electron transfer reaction rate between the second mediator and the conductive member is higher than the electron transfer reaction rate between the first mediator and the conductive member.

The first mediator preferably serves to transfer electrons to or from the enzyme, and the second mediator serves to transfer electrons to or from the first mediator.

The conductive member preferably has a porous structure.

The first mediator and the second mediator are preferably respectively at least one substance selected from metal complexes, quinones, heterocyclic compounds, nicotinamide derivatives, and flavin derivatives.

The first mediator preferably has a redox potential (reduction-oxidation potential) more negative than the redox potential (reduction-oxidation potential) of the second mediator, and is employed as an anode.

The second mediator preferably has a redox potential (reduction-oxidation potential) more positive than the redox potential (reduction-oxidation potential) of the second mediator, and is employed as a cathode.

According to another aspect of the present invention, there is provided a sensor, employing the enzyme electrode as a detection portion for detecting a substance.

According to still another aspect of the present invention, there is provided a fuel cell, employing the enzyme electrode as at least one of anode and a cathode.

According to a further aspect of the present invention, there is provided an electrochemical reactor, employing the enzyme electrode as a reaction electrode.

The process for producing an enzyme electrode having an enzyme immobilized by a carrier on a conductive member comprises a step of providing a conductive member, an enzyme, a first mediator for transfer of electrons to or from the enzyme, a second mediator for transfer of electrons to or from the first mediator, and a carrier; and a step of immobilizing the enzyme, the first mediator, and the second mediator to the conductive member.

According to the present invention, an enzyme can be immobilized on a conductive member at a high enzyme-immobilization density relative to the effective surface area of the conductive member. Use of a first mediator capable of transferring electrons rapidly to the enzyme, and a second mediator for charge transport between the first mediator and the conductive member enables rapid electron transfer to or from the enzyme, improving the current density in the enzyme electrode.

### BRIEF DESCRIPTION OF THE DRAWINGS

FIG. 1 is a schematic drawing of entrapping immobilization in an enzyme electrode.

FIG. 2 is a schematic drawing of covalent bonding immobilization in an enzyme electrode.

FIG. 3 is a schematic drawing of an enzyme electrode of the present invention.

FIG. 4 is a schematic drawing showing relation of potentials of mediators (in an anode) in the present invention

FIG. 5 is a schematic drawing showing relation of potentials of mediators (in a cathode) in the present invention FIG. 6 is a drawing for explaining a three-electrode cell.

FIG. 7 is a drawing for explaining a two-electrode cell.

FIGS. 8A, 8B, 8C and 8D show examples of the porous structures of the conductive members applicable in the present invention.

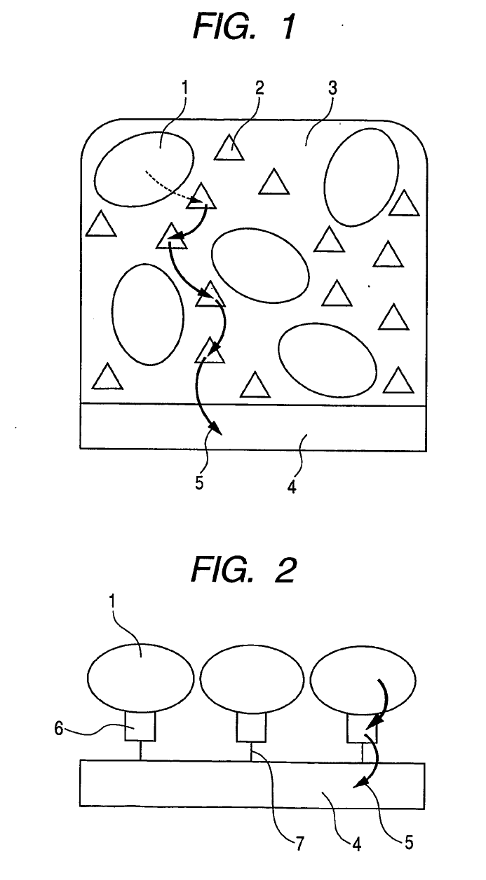

### BEST MODE FOR CARRYING OUT THE INVENTION

Preferred embodiments of the present invention are described below in detail.

The enzyme electrode of the present invention comprises a conductive member and an enzyme, wherein a first mediator and a second mediator are immobilized through a carrier onto the conductive member, the first mediator and the second mediator having different redox potentials (reduction-oxidation potentials).

In a preferred combination of the two mediators, the materials are selected such that the first mediator has a redox potential (reduction-oxidation potential) more negative than the redox potential (reduction-oxidation potential) of the second mediator (as shown in FIG. 4), and the electron transfer reaction rate between the second mediator and the conductive member is higher than the electron transfer reaction rate between the first mediator and the conductive member.

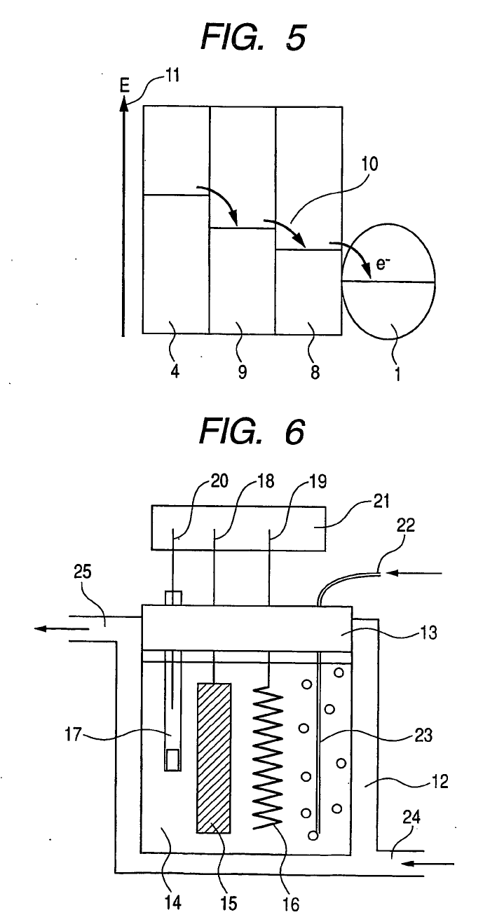

In another preferred combination of the two mediators, the materials are selected such that the first-mediator has a redox potential (reduction-oxidation potential) more positive than the redox potential (reduction-oxidation potential) of the second mediator (as shown in FIG. 5), and the electron transfer reaction rate between the second mediator and the conductive member is higher than the electron transfer reaction rate between the first mediator, and the conductive member.

The present invention is particularly useful in the cases below.

In one case, a mediator can serve to transfer electrons to or from an enzyme effectively, but cannot effectively transfer the electron to or from the mediator to a conductive member.

The magnitude relation of electron transfer rate between a mediator and a conductive member can be obtained by the comparison of standard rate constant. This constant is a measure of the kinetic facility of a redox compounds. Standard rate constant can be estimated from charge-transfer resistance that is obtained from, for example, impedance measurement. (Osaka et al. Denkikagakuhou kisosokuteimanual, koudansha scientific)

The enzyme electrode of a preferred embodiment of the present invention comprises an enzyme; at least one kind of first mediator for transferring an electron to or from the enzyme; at least one kind of second mediator for transferring an electron to or from the first mediator; a carrier immobilizing the enzyme, the first mediator, and the second mediator; and a conductive member in contact with the carrier and transferring the electron to or from the first mediator or the second mediator. The electron transfer between the enzyme and the conductive member is conducted through the first mediator at least and the second mediator, but the electron transfer may occur in the absence of the mediators, and/or in the absence of the second mediator conductive member.

The use of the carrier enables stable immobilization of the enzyme on the conductive member, and increase of the enzyme-holding density for the effective surface area of the conductive member. Further, the use of the first mediator capable of transferring electrons at a high rate to or from the enzyme and the second mediator capable of transferring electrons between the first mediator and the conductive member enables electron transfer at a high rate to or from the enzyme to improve stability of the electrode and to increase the current density in the electrode. The first mediator in the present invention transfers electrons at a higher rate to or from the enzyme than the second mediator. Consequently, the use of the first mediator results in a higher current density in the enzyme electrode than the one employing the second mediator only without the first mediator. On the other hand, the second mediator in the present invention has higher charge-transporting ability and/or gives higher electron transfer reaction rate with the electrode than the first mediator. Consequently, the use of the second-mediator gives a higher current density in the enzyme electrode than the one employing the first mediator only without the second mediator. The optimum proportions of the enzyme, the first mediator, the second mediator, and the carrier should be determined in consideration of the matters below: the proportion of the first mediator should be sufficiently high for sufficient electron transfer to or from the enzyme; the proportion of the second mediator should be high for sufficient charge transport between the enzyme and the conductive member; the proportion of the carrier is sufficiently high for sufficient retention of the enzyme and the mediator; and the immobilization density should be sufficiently high. For example, for a unit weight of the enzyme, the first mediator weighs preferably from 0.0001 to 1, more preferably from 0.000.2 to 0.1; the second mediator weighs preferably from 0.01 to 2, more preferably from 0.3 to 1; and the carrier weighs preferably from 0.05 to 3, more preferably from 0.3 to 1.

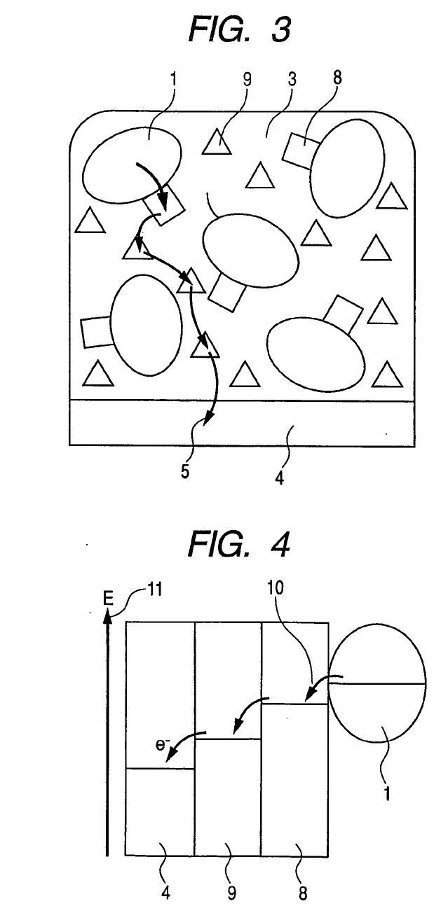

FIG. 3 illustrates an immobilization state of an enzyme, a first mediator, and a second mediator on an enzyme electrode of the present invention. In this example, enzyme 1, first mediator 8, and second mediator 9 are entrapped in layer 3 of a carrier provided on the surface of conductive member 4 (conducting substrate). An example of a flow of electrons is shown by an arrow mark.

In this enzyme electrode, in use of this enzyme electrode as a positive electrode or a negative electrode, for effective electron transfer between the first mediator (for high-speed electron transfer to or from the enzyme) and the second mediator (for charge transport between the first mediator and the conductive member), the first mediator has a redox potential (reduction-oxidation potential) more negative than that of the second mediator when the enzyme electrode is utilized as an anode (FIG. 4), whereas the first mediator has a redox potential (reduction-oxidation potential) more positive than that of the second mediator when the enzyme electrode is utilized as a cathode (FIG. 5).

A sensor, which is a preferred embodiment of the present invention, employs the enzyme electrode as a sensing part for detecting a substance. In a typical constitution, an enzyme electrode as a working electrode, a reference electrode, and a counter electrode are employed in a set. A catalytic current is detected by the enzyme electrode (by function of the enzyme immobilized on the electrode) to detect a substance in the liquid in contact with the electrodes. FIG. 6 shows a specific example of the constitution of the sensor. In FIG. 6, the sensor comprises anode 15, platinum wire 16, and optionally Ag/AgCl reference electrode 17, and the respective electrodes are connected through lead wires 18-20 to a potentiostat. This sensor is placed in electrolyte solution 14 in water-jacketed cell 12 closable with a cover 13. A substrate in the electrolyte solution can be detected by applying a potential to the working electrode and measuring the steady-state current. When the measurement should be conducted in an inert atmosphere, an inert gas like nitrogen is introduced from gas inlet 22 of gas tube 23. The temperature can be controlled by a temperature-controlling liquid by introduction of controlling liquid inlet 24 to temperature-controlling liquid outlet 25.

The constitution of the sensor of the present invention is not limited insofar as the enzyme electrode can conduct detection. This sensor has high substrate selectivity owing to the enzyme employed as the electrode reaction catalyst, and has high stability and high current density owing to use of the carrier and plural mediators, whereby the sensor can be used repeatedly for a long term, the sensor can be simplified, or the sensing part can be miniaturized. The enzyme electrode may be in a board shape, or a single or multiple layer shape. This sensor is capable of detecting a substance corresponding to the substrate of the enzyme of the enzyme electrode, being useful, for example, as a glucose sensor, a fructose sensor, a galactose sensor, an amino acid sensor, an amine sensor, a cholesterol sensor, an alcohol sensor, a lactic acid sensor, an oxygen sensor, a hydrogen peroxide sensor, or the like. More specific application examples are a sensor for measuring a glucose concentration or lactic acid concentration in blood, a sensor for measuring a sugar concentration in a fruit, and a sensor for measuring an alcohol concentration in exhaled breath.

A fuel cell, a preferred embodiment of the present invention, employs an enzyme electrode as at least one of the anode and the cathode. In this embodiment also, the enzyme electrode may be in a board shape, a monolayer shape, or a multilayer structure. In the multilayer structure, anodes and cathodes are arranged as predetermined. In a typical constitution, the fuel cell has a reaction layer for holding an electrolyte solution containing a fuel source material, and an anode and a cathode placed at a predetermined interval, at least one of the anode and the cathode being the enzyme electrode of the present invention. The fuel cell may be of a type in which an electrolyte solution is replenished or circulated, or may be of a type in which an electrolyte solution is neither replenished nor circulated. The fuel cell is not limited in the fuel, the structure, the function, and so forth, insofar as the enzyme electrode is usable. The enzyme, the catalyst constituting the enzyme electrode, has a high substrate selectivity in comparison with a noble metal catalyst (e.g., platinum) employed generally in electrochemical fields. Therefore, the reaction substances on the anode and the cathode need not be separated by a partition, which can simplify the device. This fuel cell can generate a high driving voltage by oxidation-reduction of a substance at a low overvoltage owing to a characteristic high activity of the employed enzyme as the catalyst for the electrode reaction. The fuel cell can have a long life and give high output by using a carrier and plural mediator.

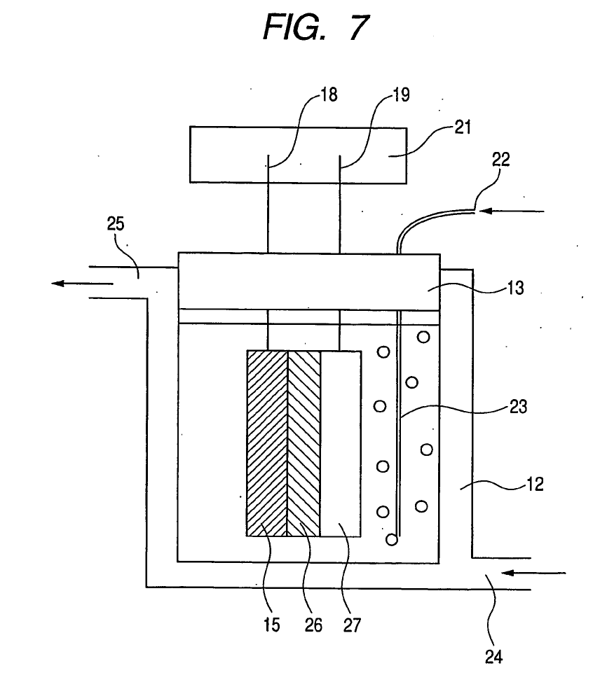

FIG. 7 shows an example of the fuel cell. The fuel cell has a constitution similar to the one shown in FIG. 6 of the substrate detecting sensor. Therefore, in FIG. 6 and I₁ the same reference numerals are used for denoting the correspondent conductive members. The fuel cell is constituted by replacing the sensor in FIG. 6 by an electrode unit constituted of anode 15 and cathode 27 laminated with interposition of a porous polypropylene film. The fuel cell is driven by introducing gaseous oxygen through tube 23 into the cell.

An electrochemical reactor, a preferred embodiment of the present invention, can give high substrate selectivity and high catalytic ability characteristic of the enzyme utilized in the electrode reaction catalyst as well as the quantitativeness characteristic to the electrochemical reaction. Therefore, the reactor is capable of controlling the reaction quantitatively with high selectivity, and high efficiency. The reactor can have a long life and a high power output with high stability and a high current density by use of a carrier and plural mediators. In this embodiment also, the enzyme electrode may be in a board shape, a monolayer shape, or a multilayer structure. In a typical constitution, a pair of electrodes and an optionally provided reference electrode are placed in a reaction vessel, and an electric current is allowed to flow between the pair of electrodes to cause an electrochemical reaction of a substance in the reaction solution to obtain an intended reaction product or decomposition product: at least one of the pair of electrodes is an enzyme electrode of the present invention. The kind of the reaction solution, the reaction conditions, and the constitution of the reactor are not specially limited, insofar as the enzyme electrode is usable. For example, the reactor is useful for obtaining an intended reaction product or decomposition product by an oxidation-reduction reaction.

The electrochemical reactor can cause reaction of a substance corresponding to the substrate of the enzyme used in the enzyme electrode. For example, the reactor is useful for oxidation of glucose, fructose, galactose, amino acids, amines, cholesterol, alcohols, lactic acid, and so forth; reduction of oxygen, hydrogen peroxide, and so forth; and the like reactions. More specific application examples include selective oxidation of cholesterol in the presence of ethanol, and reduction of oxygen at a low overvoltage.

The carrier in the present invention serves at least to immobilize the enzyme to the conductive member. The carrier includes (1) polymer compounds, (2) inorganic compounds, and (3) organic compounds, the compounds having a covalent bonding site in the molecule and being capable of bonding an enzyme to the conductive member (the carrier may have additionally a function of bonding two enzymes). The carrier contains at least one of the above three types of compounds. To immobilize the enzyme to the electrode, the carrier has preferably an electric charge opposite to the surface charge of the enzyme under the electrode driving conditions. The carrier may be ones capable of holding the enzyme by covalent bonding, electrostatic interaction, spatial trapping, or a like action to hold the enzyme stably at a high density in comparison with retention of the enzyme by physical adsorption to the electrode or to a binder polymer for caking the electrode. The polymer compounds useful as the carrier include electroconductive polymers such as polyacetylenes, polyarylenes, polyarylene-vinylenes, polyacenes, polyarylacetylenes, polydiacetylenes, polynaphthalenes, polypyrroles, polyanilines, polythiophenes, polythienylene-vinylenes polyazulenes, and polyisothianaphthenes; and other kind of polymers such as polystyrenesulfonic acids, polyvinyl sulfate, dextran sulfate, chondroitin sulfate, polyacrylic acid, polymethacrylic acid, polymaleic acid, polyfumaric acid, polyethylenimine, polyallylamine hydrochloride, polydiallyldimethylammoniur α chloride polyvinylpyridine, polyvinylimidazole, polylysine, deoxyribonucleic acid, ribonucleic acid, pectin, silicone resins, cellulose, agarose, dextran, chitin, polystyrene, polyvinyl alcohol, and nylons.

The inorganic compounds useful as the carrier include metal chalcogenide compounds containing at least one element selected from the group of In, Sn, Zn, Ti, Al, Si, Zr, Nb, Mg, Ba, Mo, W, V, and Sr.

The organic compounds, being useful as the carrier, and having a covalent bonding site in the molecule and being capable of bonding an enzyme to the conductive member, and/or the two enzymes include compounds having at least one functional group selected from hydroxyl, carboxyl, amino, aldehydo, hydrazino, thiocyanato, epoxy, vinyl, halogeno, acid ester groups, phosphato, thiol, disulfido, dithiocarbamato, dithiophosphato, dithiophosphnato, thioether groups, thiosulfato, and thiourea groups. Typical examples are glutaraldehyde, polyethylene glycol diglycidyl ether, cyanuric chloride, N-hydroxysuccinimide esters, dimethyl-3,3′-dithiopropionimidate hydrochloride, 3,3′-dithio-bis (sulfosuccinimidyl propionate), cystamine, alkyl dithiols, biphenylene dithiols, and benzene dithiols.

The conductive member in the present invention serves to hold the enzyme, the mediator, and the carrier for immobilizing the enzyme and the mediators, and to connect the enzyme electrode electrically to an external circuit on driving the enzyme electrode. This conductive member should have electroconductivity, sufficient rigidity during storage and measurement, and should be electrochemically stable under electrode driving conditions. The constituting material of the conductive member includes electroconductive materials such as metals, electroconductive polymers, metal oxides, and carbonaceous materials.

The metal includes those containing at least one element of Au, Pt, Ag, Co, Pd, Rh, Ir, Ru, Os, Re, Ni, Cr, Fe, Mo, Ti, Al, Cu, V, Nb, Zr, Sn, In, Ga, Mg, Pb, Si, and W. The metal may be an alloy, or a plating metal.

The electroconductive polymers include those containing at least one of polyacetylenes, polyarylenes, polyarylene-vinylenes, polyacenes, polyarylacetylenes, polydiacetylenes polynaphthalenes, polypyrroles, polyanilines, polythiophenes, polythienylenevinylenes, polyazulenes, and polyisothianaphthenes.

The metal oxides may contain other conductive material for improving the conductivity thereof or imparting conductivity. The metal oxides include oxides of metals containing at least one of In, Sn, Zn, Ti, Al, Si, Zr, Nb, Mg, Ba, Mo, W, V, and Sr. The electroconductive materials include metals electroconductive polymers, and carbonaceous materials. The carbonaceous material may contain other electroconductive material for improving or imparting the electroconductivity. The carbonaceous materials include graphite, carbon black, carbon nanotubes, carbon nanohorns, fullerene compounds, and derivatives thereof.

The electric conductivity of the conductive member applicable to the present invention ranges from 0.1 to 700000 S/cm, preferably from 1 to 100000 s/cm, more preferably from 100 to 100000 S/cm. (Incidentally, “S” denotes Siemens, a reciprocal of ohm (1/Ω).) Even when, the enzyme electrode has a porous structure, the conductive member has preferably the electric conductivity in the above range.

The conductive member has preferably a structure having numerous voids communicating with each other, the voids communicating with the outside of the conductive member directly or through other voids and being capable of immobilizing an enzyme, a first mediator, and a second mediator therein. The conductive member having numerous voids communicating with the outside has preferably a structure in which void-partitioning walls are formed integrally from the constitution material, or the void-partitioning walls are bonded tightly with each other. The material for constituting the conductive member includes the above-mentioned conductive materials of metals, polymers, metal oxides, and carbonaceous substances.

The numerous voids in the conductive member are interconnected together in one-, two-, or three-dimensionally. The interconnection of the voids may be of two or more types. The one-dimensional void interconnection is exemplified by columnar voids; the two-dimensional void interconnection is exemplified by net-like voids; and the three-dimensional void interconnection is exemplified by sponge-like voids, interstices formed in aggregation of small particles, and voids, in a structural material prepared by use of the above material as a template. The voids size should be large for introduction of the enzyme and flow and diffusion of the substrate substance, but should be small within the range for obtaining a sufficient ratio of the effective void surface area to the projected area of the conductive member. The average void size ranges, for example, from 5 nm to 500 μm, more preferably from 10 nm to 100 μm. The void-containing conductive member should have a small thickness for introduction of the enzyme into the deep interior of the conductive member and flow and diffusion of the substrate through the conductive member, but should have thickness within the range for obtaining a sufficient ratio of the effective void surface area to the projection area of the conductive member. The thickness of the void-containing conductive member ranges, for example, from 100 nm to 1 cm, more preferably from 1 μm to 5 mm. The ratio of the effective surface area to the projected area of the void-containing conductive member should be sufficiently large, for example, the ratio being 10 or more, more preferably 100 or more. The porosity of the void-containing conductive member should be sufficiently large for obtaining a high ratio of the effective void surface area to the projected area of the conductive member, and be large within the range for enabling introduction of the enzyme and the carrier and flow and diffusion of the substrate substance, but should be small for achieving the sufficient mechanical strength. The porosity ranges, for example, from 20% to 99%, more preferably from 30% to 98%. The porosity of the conductive member having an enzyme immobilized therein should be large for flow of the electrolyte solution and diffusion of the substrate substance, but should be small by filling of the enzyme. The porosity ranges for example, from 15% to 98%, more preferably from 25% to 95%.

The constituting material for the void-containing conductive member includes foamed metals, electrodeposited metals, electrolytic metals, sintered metals, fibrous metals, and metals corresponding to two or more of the above kinds of metals.

The void-containing polymer can be produced by any of the processes for manufacture of a porous resin. In one process, a conductive polymer is molded in the presence of a void template into an intended shape, and thereafter the material of the void template is removed. In another process, a template for the voids is placed in a prepolymer, the prepolymer is polymerized into a conductive polymer, and thereafter the material of the template is removed. In a still another process, a layer is formed from particles for constituting a void template, a polymer is filled into the interstice of the particle layer, and thereafter the particles are removed from the layer. In a still another process, a layer is formed from particles for constituting a void template, a prepolymer is filled into the interstice of the particle layer, the prepolymer is polymerized to form a polymer layer, and thereafter the particles are removed from the layer.

The void-containing conductive member constituted of a metal oxide having numerous voids can be obtained by a method including electro-deposition, sputtering, sintering, chemical vapor deposition (CVD), electrolysis, and so forth, and combinations thereof. Otherwise, the conductive member having numerous voids can be obtained from fibers or particles of graphite, carbon black, carbon nanotubes, carbon nanohorns, a fullerene compound, or a derivatives thereof by forming the material into an intended shape and sintering the formed material.

In the conductive member, the voids may have a Uniform size (or uniform porosity) in the thickness direction of the porous structure, or the voids may have a gradient distribution of the size or porosity.

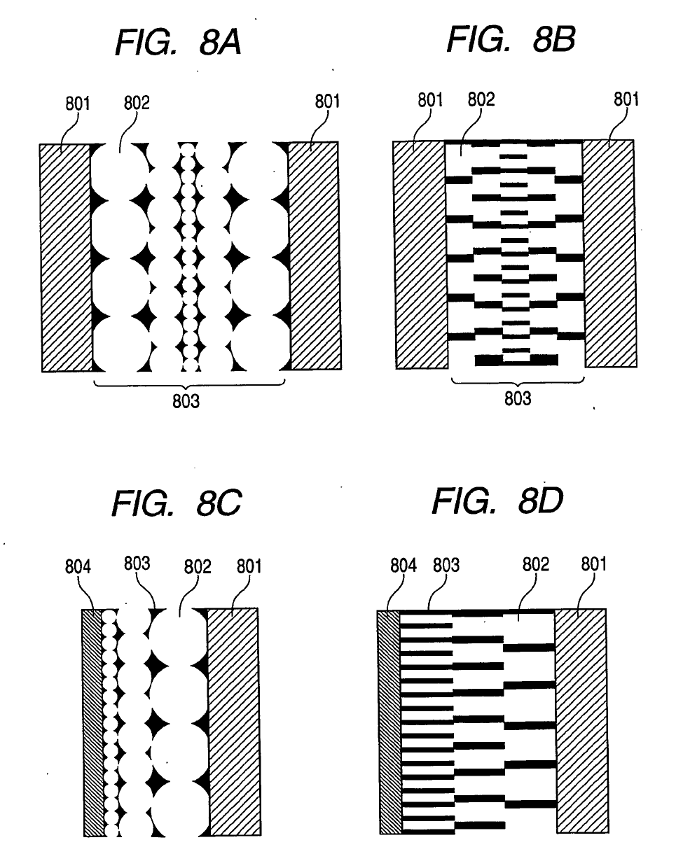

FIGS. 8A to 8D illustrates porous structures of the conductive member. In the drawings, the numerals denote the followings: 801, an electrolyte layer; 802, a pore; 803, a conductive member; 804, supporting substrate optionally employed. As shown in the drawings, the sizes of the pores in the conductive member are preferably larger at the electrolyte layer side and smaller in the inside.

In other words, in the conductive porous conductive member employed in the present invention, the pore sizes are preferably larger at the surface side of the conductive porous conductive member than those at the interior thereof. The pore size ratio is preferably 2 or more, more preferably 4 or more, still more preferably 10 or more, but is not larger than 1000.

The porosity may be the same between the regions of different pore sizes. More preferably, the pore sizes and the porosities in the conductive member are both larger in the electrolyte layer side, and smaller in the interior.

The pore size and porosity of the porous conductive member can be measured by nitrogen gas adsorption measurement (BET method (Brunauer-Emmett-Teller method)), for example by AUTOSORB-I (Quantachrome Instruments Co.). The pore sizes on the surface of the conductive member can be estimated by measuring the pore sizes of a certain number of pores (e.g., about 50-300 pores) in SEM photograph (scanning electron microscope photograph).

The mediator in the present invention promotes electron transfer through enzyme/mediator/conductive member. This mediator may be chemically bonded to the carrier and/or the enzyme. The mediator is exemplified by metal complexes, quinones, heterocyclic compounds, nicotinamide derivatives, and flavin derivatives. The metal complexes include those having as the central metal at least one element selected from Os, Fe, Ru, Co, Cu, Ni, V, Mo, Cr, Mn, Pt, Rh, Pd, Mg, Ca, Sr, Ba, Ti, Ir, Zn, Cd, Hg, and W. The ligands of the metal complexes are exemplified by those containing an atom of nitrogen, oxygen, phosphorus, sulfur, or carbon and capable of forming a complex through the above atom with the central metal; and those having a cyclopentadienyl ring as the skeleton. The ligand includes pyrrole, pyrazole, imidazole, 1,2,3- or 1,2,4-triazole, tetrazole, 2,2′-biimidazole, pyridine, 2,2′-bithiophene, 2,2′-bipyridine, 2,2′:6′2″-terpyridine, ethylenediamine, porphyrin, phthalocyanine, acetylacetone, quinolinol, ammonia, cyan, ions, triphenylphosphine oxide, and derivatives thereof. The quinones as the mediator include quinone, benzoquinone, anthraquinone, naphthoquinone, pyrroloquinolinequinone (PQQ), tetracyanoquinonedimethane, and derivatives thereof. The heterocyclic compounds as the mediator include phenazine, phenothiazine, biologen, and derivatives thereof. The nicotinamide derivatives as the mediator include nicotinamide adenine dinucleotide (NAD), and nicotinamide adenine dinucleotide phosphate. The flavin derivatives as the mediator include flavin adenine dinucleotide (FAD).

The enzyme in the present invention serves to catalyze an oxidation-reduction reaction, and may be a combination of plural enzymes. The enzymes include glucose oxidase, galactose oxidase, bilirubin oxidase, pyruvate oxidase, D- or L-amino acid oxidase, amine oxidase, cholesterol oxidase, choline oxidase, xanthine oxidase, sarcosine oxidase, L-lactate oxidase, ascorbate oxidase, cytochrome oxidase, alcohol dehydrogenase, glutamate dehydrogenase, cholesterol dehydrogenase, aldehyde dehydrogenase, glucose dehydrogenase, fructose dehydrogenase, sorbitol dehydrogenase, lactate dehydrogenase, malate dehydrogenase, glycerol dehydrogenase, 17B-hydroxysteroid dehydrogenase, estradiol-17B dehydrogenase, amino acid dehydrogenase, glyceraldehyde-3-phosphate dehydrogenase, 3-hydroxysteroid dehydrogenase, diaphorase, catalase, peroxidase, glutathione reductase, NADH-cytochrome b5 reductase, NADPH-adrenodoxin reductase, cytochrome b5 reductase, adrenodoxin, reductase, and nitrate reductase.

The substrate substances for the electrode-constituting enzymes are compounds corresponding to the respective enzymes, including organic matters, oxygen, hydrogen peroxide, water, and nitrate ions. The organic matters include sugars, alcohols, carboxylic acids, quinones, nicotinamide derivatives, and flavin derivatives. The sugars include polysaccharides such as cellulose, and starch.

In the step of immobilizing the carrier in the present invention, uniformity is important in the immobilization of the carrier on the conductive member. In the step of immobilizing the carrier, the enzyme and/or the mediator may be concurrently immobilized. The step of immobilizing the carrier may be a step of immersing a void-containing conductive member in a solution or dispersion liquid of the carrier; a step of applying, injecting, or spraying a solution or dispersion liquid of the carrier onto a void-containing conductive member; or a step of applying, injecting, dr spraying a solution or dispersion liquid of the carrier precursor onto a void-containing conductive member and thereafter hydrolyzing, polymerizing, or crosslinking the precursor for immobilization.

In the drawings, the reference numerals denote the followings: 1, an enzyme; 2 and 6, a mediator; 3 and 7, a carrier; 4, a conductive member; 5, a flow of a charge; 8, a first mediator; 9, a second mediator; 10, a flow of an electron; 11, a potential (the arrow indicates the negative direction); 12, a water-jacketed cell; 13, a cover for the water-jecketed cell; 14, an electrolysis solution; 15, an anode; 16, a platinum wire; 17, an Ag/AgCl reference cell; 18, an anode lead; 18, a cathode lead; 20 a reference electrode lead; 21, a potentiostat; 22, a gas inlet; 23, a gas tube; 24, a temperature-controlling water inlet; 25, a temperature-controlling outlet; 26, porous polypropylene film; 27, a cathode.

### EXAMPLES

The present invention is explained below in more detail by reference to examples without limiting the invention.

Firstly, the processes for preparation of the conductive members for use for the enzyme electrode of the present invention are described.

### Preparation Example 1

A commercial silica colloid dispersion liquid (Nissan Chemical Ind.; average particle size: 100 nm) is employed. The dispersion medium of the dispersion liquid is replaced by ethanol. A cleaned gold substrate (1 cm square, 0.3 mm thick, Nilaco) is allowed to stand in the dispersion liquid. The ethanol is allowed to evaporate at 30° C. to obtain a porous film constituted of silica spheres. This process is repeated several times to increase the thickness of a porous film constituted of silica spheres (100 nm thick). The film is heated at 200° C. for three hours, and then washed with ethanol. In a three-electrode cell, by use of this porous film as the working electrode, a platinum electrode as the counter electrode, and an Ag/AgCl electrode as the reference electrode, electrolytic polymerization is conducted in a solution of 0.1M 3,4-ethylenedioxythiophene and 0.1M lithium perchlorate in acetonitrile at a potential of 1.1 V (vs Ag/AgCl) by control with a potentiostat. The time of the polymerization is controlled by monitoring the electrolysis current profile to obtain a film in a thickness nearly equivalent to the silica sphere porous film thickness. After the electrolytic polymerization, the film is immersed in a 20% hydrofluoric acid solution for two days to remove the silica spheres to obtain a conductive member (100 μm thick) constituted of electroconductive poly(3,4-ethylenedioxythiphene) containing numerous voids.

### Preparation Example 2

Needle-shaped indium tin oxide (ITO, Sumitomo Metal Mining Co.; length: 30-100 nm; aspect ratio: 10 or higher) is dispersed in terpinol, and the viscosity is adjusted by addition of ethylcellulose to obtain an ITO paste. This ITO paste is applied on a cleaned gold substrate (1 cm square, 0.3 mm thick, Nilaco) by screen process printing, and is sintered at 450° C. for one hour to obtain a porous ITO sintered electrode (100 μm thick). Further thereon, ITO is deposited by plasma chemical vapor phase deposition (CVD) in a thickness of about 10 nm to obtain a conductive member constituted of ITO having numerous voids.

### Preparation Example 3

Natural particulate graphite (particle size: 11 μm) is mixed with polyvinylidene fluoride in an amount of 10 wt % of the particulate graphite. N-methyl-2-pyrrolidone is added thereto to solve the polyvinylidene fluoride. The blended graphite paste is molded into a sheet of 11.3 mm diameter and 0.5 mm thick. The film is dried at 60° C., heated to 240° C., and further vacuum-dried at 200° C. Thereby a conductive member is obtained which is constituted of many graphite particles bonded together and has interconnected numerous voids in the structure.

### Preparation Example 4

N⁶-(2-aminoethyl) FAD shown by Chemical Formula (1) is prepared through the process shown below. To an aqueous 10% FAD solution, is added an equimolar amount of ethylenimine, The pH is adjusted to 6-6.5. The mixture is allowed to react at 50° C. for 6 hours. The reaction solution is cooled, and is added into ethanol on an ice bath to cause precipitation. The precipitate is collected and is purified by anion exchange chromatography and reversed-phase high-speed chromatography to obtain purified N⁶-(2-aminoethyl) FAD shown by Chemical Formula (1).

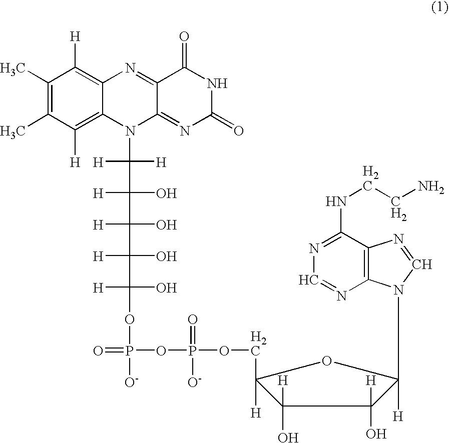

### Preparation Example 5

The process for synthesis of the ferrocene derivative shown by Chemical Formula (2) is described below. A 4.7 g portion of β-aminocaproic acid is dissolved in 200 mL of DMF. Thereto, is added a solution of 2.1 g ferrocene carbaldehyde in 100 mL of DMF. The mixture is stirred at 100° C. for one hour. Thereto is added 1 g of sodium boron hydride saturated in water. The mixture is stirred at room temperature for one hour. The solvent is evaporated off under a reduced pressure. The evaporation residue is treated by a silica column with a solvent of dichloromethane/methanol (10/1) to obtain the ferrocene derivative compound represented by Chemical Formula (2). The compound is identified by ¹H-NMR.

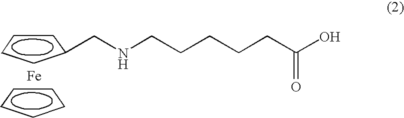

### Preparation Example 6

The osmium complex-containing polymer shown by Chemical Formula (3) is synthesized through the steps below. To 6 mL of 1-vinylimidazole, is added 0.50 g of azobisisobutyronitrile. The mixture is allowed to react in an argon atmosphere at 70° C. for 2 hours. After air-cooling of the reaction mixture, the formed precipitate is dissolved in methanol, and the solution is added dropwise to acetone with violent agitation. The formed precipitate is collected by filtration to obtain poly-1-vinylimidazole Separately, to 1.2 g of 4,4-dimethyl-2,2′-bipyridine and 1.4 g of ammonium hexachloroosmate, is added 5 mL of ethylene glycol. The mixture is allowed to react in a nitrogen atmosphere at 140° C. for −24 hours. The reaction solution is cooled by air, and is filtered to remove impurity. The filtrate is evaporated at a reduced pressure to obtain Os (4,4′-dimethyl-2,2′-bipyridine) 2 dichloride salt. To 200 mL of ethanol, are added 0.13 g of Os (4,4′-dimethyl-2,2′-bipyridine) 2 dichloride salt and 0.20 g of polyvinylimidazole. The mixture is refluxed in a nitrogen atmosphere for 3 days. The reaction solution is filtered, and the filtrate is added dropwise to 1 L of diethyl ether with violent agitation. The formed precipitate is recovered and dried to obtain the osmium complex polymer shown by Chemical Formula (3). The compound is identified by elemental analysis.

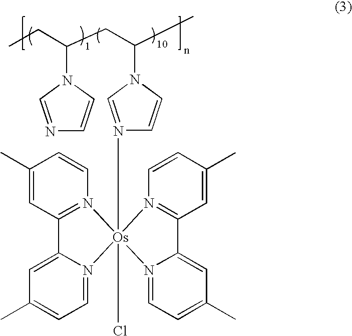

### Preparation Example 7

The ferrocene-containing polymer shown by Chemical Formula (4) is prepared through the steps below. To 3.0 mL Of toluene, are added 1.6 g of 1-vinylimidazole, 0.71 g of 1-vinylferrocene, and 6.2 mg of azobisisobutyronitrile. The mixture is allowed to react in an argon atmosphere at 70° C. for 2 hours. The reaction solution, after air-cooling, is added dropwise to 1 L of diethyl ether. The formed precipitate is collected by filtration to obtain the ferrocene-containing polymer shown by Chemical formula (4).

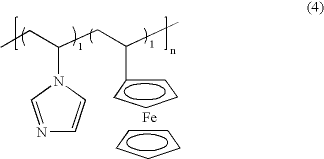

### Preparation Example 8

The process for synthesis of the complex polymer shown by Chemical Formula (5) is described below.

To 100 g of an aqueous 40% glyoxal solution, is added dropwise 370 mL of an aqueous concentrated ammonia solution on an ice bath. The mixture is stirred at 45° C. for 24 hours, and is air-cooled. The formed precipitate is collected by filtration, and vacuum-dried at 50° C. for 24 hours to obtain 2,2′-biimidazole. This compound is identified by silica-gel thin-layer chromatography (methanol/chloroform (10%/90%)). To a solution of 4.6 g of 2,2′-biimidazole in 100 mL of N,N′-dimethyl formamide (DMF), is added 2.7 g of sodium hydride in a nitrogen atmosphere on an ice bath. The mixture is stirred at room temperature for one hour. Thereto, a solution of 12.8 g of methyl p-toluenesulfonate in 5 mL of DMF is added dropwise in 20 minutes, and the mixture is stirred at room temperature for 4 hours. The solvent is evaporated under vacuum at 50° C. The evaporation residue is washed with 50 mL of hexane, and vacuum-dried at 160° C. to obtain N,N′-dimethyl-2,2-biimidazole in a colorless transparent crystalline state. The obtained product is identified by ¹H-NMR.

To a solution of 10 g 2,2′-biimidazole in 100-mL of DMF, is added 3.3 g of sodium hydride on an ice bath in a nitrogen atmosphere. The mixture is stirred on an ice bath for one hour. Thereto, 4.6 mL of methyl iodide is added dropwise, and the mixture is stirred on an ice bath for 30 minutes and at room temperature for 12 hours. The reaction solution is poured into 300 mL of ethyl acetate. The mixture is filtered, and the solvent is evaporated from the filtrate under a reduced pressure and vacuum. The evaporation residue is dissolved in boiling ethyl acetate, and the solution is filtered. The filtered ethyl acetate solution is refluxed again. Thereto 300 mL of hexane is added for saturation. The solution is kept in a refrigerator for 12 hours for crystal growth. The crystalline matter is collected by suction filtration, and recrystallized from ethyl acetate/hexane to obtain N-methyl-2,2′-biimidazole. This compound is identified by ¹H-NMR.

A 1 g portion of N-methyl-2,2′-biimidazole is dissolved in 80 mL of DMF. Thereto, 0.32 g of sodium hydride is added in a nitrogen atmosphere. The mixture is stirred on an ice bath for one hour. Thereto 2.5 g of N-(β-bromohexyl)phthalimide and 1.0 g of sodium iodide are added gradually. The mixture is stirred in a nitrogen atmosphere at 80° C. for 24 hours. The mixture is cooled to room temperature, and 150 mL of water is added thereto. The mixture is extracted twice with 150 mL portions of ethyl acetate. The ethyl acetate solution is washed with an aqueous sodium chloride solution and dried over sodium sulfate, and is evaporated under a reduced pressure. The residue is purified by a neutral alumina column (ethyl acetate/hexane 10-40%) to obtain N-methyl-N′-(β-phthalimidohexyl)-2,2′-biimidazole. This product is identified by ¹H-NMR.

A 2.5 g portion of N-methyl-N′-(6-phthalimidohexyl)-2,2′-biimidazole is dissolved in 25 mL of ethanol. Thereto 0.39 g of hydrogenated hydrazine is added. The mixture is refluxed for 2 hours, cooled to room temperature, and filtered. The solution is transferred to a silica gel column with ethanol. The product is recovered by a 10% ammonia solution in acetonitrile, and the solution is evaporated under a reduced pressure to obtain N-(6-aminohexyl)-N′-methyl-2,2′-biimidazole. This product is identified by ¹H-NMR.

In 40 mL of ethylene glycol, 1.1 g of N-methyl-2,2′-biimidazole and 1.4 g of ammonium hexachloroosmate are dissolved. The solution is stirred in a nitrogen atmosphere at 140° C. for 24 hours. Thereto, is added a solution of 0.8 g of N-(6-aminohexyl)-N′-methyl-2,2′-biimidazole in 5 mL of ethylene glycol. The solution is stirred further for 24 hours, cooled to room temperature, and filtered. The filtrate is diluted with 200 mL of water, and stirred with 40 mL of an anion exchange resin (DOWEX® 1×4) in the atmosphere for 24 hours. The solution is poured gradually into a solution of 10.2 g of ammonium hexafluorophosphate in 150 mL of water. The precipitate is collected by filtration by suction, and dissolved in acetonitrile and reprecipitated by an aqueous ammonium hexafluorophosphate solution. The obtained matter is washed with water, and vacuum-dried at 45° C. for 24 hours to obtain osmium (III) (N,N′-dimethyl-2,2′-biimidazole) 2(N-(6-aminohexyl)-N′-methyl-2,2′-biimidazole) hexafluorophosphate salt. This product is identified by elemental analysis.

To 150 mL of DMF, are added 20 g of polyvinylpyridine (average molecular weight: 150,000) and 5.6 g of 6-bromohexane. The mixture is stirred at 90° C. with a stirrer for 24 hours, and cooled to room temperature. The cooled mixture is poured gradually into 1.2 L of ethyl acetate with violent agitation. Then the solvent is removed by decantation, and the remaining solid matter is dissolved in methanol. The solution is filtered, and evaporated to a solvent volume of about 200 mL. The formed product is reprecipitated with 1 L of diethyl ether. The product is vacuum-dried at 50° C. for 24 hours, pulverized, and further dried for 48 hours to obtain poly(4-(N-(5-carboxypentyl)pyridinium)-co-4-vinylpyridine).

In 10 mL of DMF, 0.52 g of the poly(4-(N-(5-carboxypentyl) pyridinium)-co-4-vinylpyridine) is dispersed, and thereto 0.18 g of O—(N-succinimidyl)-N,N,N′,N″-tetramethyluronium tetrafluoroborate (TSTU) is added. The mixture is stirred for 15 minutes. Thereto 0.1 mL of N,N-diisopropylethylamine is added, and the mixture is stirred for 8 hours. Thereto, 0.89 g of poly(4-(N-(5-carboxypentyl) pyridinium)-co-4-vinylpyridine) is added and the mixture is stirred for 5 minutes. Further thereto, 0.1 mL of N,N-diisopropylethylamine is added and the mixture is stirred at room temperature for 24 hours. The resulting mixture is added to 200 mL of ethyl acetate. The formed precipitate is collected by filtration, and is added to 30 mL of acetonitrile. Thereto 40 mL of DOWEX® 1×4, and 100 mL of water are added, and the mixture is stirred for 36 hours to dissolve the polymer. The solution is filtered by suction, and is concentrated to a volume of 50 mL. The concentrated matter is extruded through a (mol wt 10000) cutoff filter (Millipore) at a nitrogen pressure of 275 kPa. Further, the extruded matter is passed with water as the solvent through a DOWEX® 1×4 column, and dialyzed in water. Thereby the polymer (chloride salt) of Chemical Formula (1) is obtained.

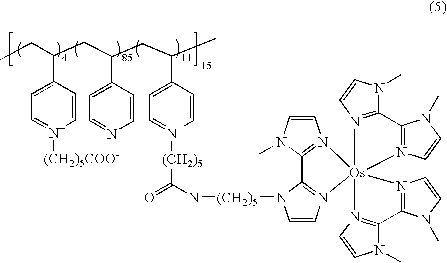

### Preparation Example 9

The process for synthesis of the complex polymer shown by Chemical Formula (6) is described below.

To 6 mL of 1-vinylimidazole, is added 0.5 g of azobisisobutylonitrile. The mixture is allowed to react in an argon atmosphere at 70° C. for 2 hours. The reaction solution is air-cooled. The formed precipitate is dissolved in methanol. The solution is added dropwise into acetone with violent agitation. The precipitate is collected by filtration to obtain poly-1-vinylimidazole. Separately, 0.76 g of 2,2′:6′2″-terpyridine and 1.42 g of ammonium hexachloroosmate are added to 5 mL of ethylene glycol, and the mixture is refluxed in an argon atmosphere for one hour. To this solution, 0.60 g of 4,4′-dimethyl-2,2′-bipyridine is added. The mixture is refluxed for 24 hours. The reaction solution is air-cooled. Impurity is removed by filtration. The filtrate is evaporated to remove the solvent to obtain osmium (2,2′:6 2″-terpyridine) (4,4′-dimethyl-2,2′-bipyridine) chloride salt.

A 200 mL portion of ethanol is added to 0.38 g of osmium(2,2′:6′2″-terpyridine) (4,4′-dimethyl-2,2′-bipyridine) chloride salt and 0.2 g of polyvinylimidazole. The mixture is refluxed in a nitrogen atmosphere for three days. The reaction mixture is filtered, and then the filtrate is added dropwise into 1 L of diethyl ether with violent agitation. The formed precipitate is recovered and dried to obtain the osmium complex represented Toy Chemical Formula (6). The compound is identified by elemental analysis.

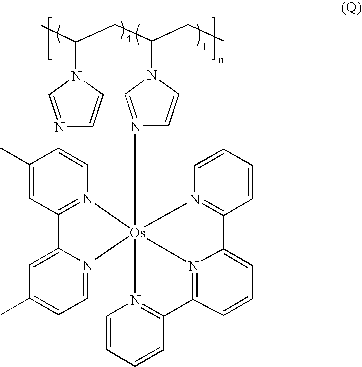

### Preparation Example 10

An FAD-free apoglucose oxidase is prepared through the process below. Glucose oxidase (Aspergillus niger) is dissolved in 3 mL of a 0.25M sodium phosphate buffer solution (pH 6) containing 30% glycerol. This solution is cooled to 0° C., and the pH thereof is adjusted to 1.7 by addition of a 0.025M sodium phosphate buffer solution-sulfuric acid solution containing 30% glycerol (pH 1.1). This solution is allowed to pass through a Sephadex® G-25 column with a 0.1M sodium phosphate solution (pH: 1.7) containing 30% glycerol, and the intended fraction is recovered by monitoring with light of a wavelength of 280 nm. Dextran-coated charcoal is added to the recovered solution. The solution, after adjustment of pH to 7 by addition of a 1M sodium hydroxide solution, is stirred at 4° C. for one hour. The resulting solution is centrifuged, passed through a 0.45 μm filter, and dialyzed by use of a 0.1M sodium phosphate buffer solution to obtain the apoglucose oxidase.

### Preparation Example 11

An FAD-free apo-D-amino acid oxidase is prepared through the process below. D-amino acid oxidase (swine kidney) is dialyzed against a 0.1M pyrophosphate buffer solution containing 1M potassium bromide and 3.0 mM ethylenediamine tetraacetate at 4° C. for 2 days with four liquid-exchange's. After confirmation of disappearance of the color of holo-D-amino acid oxidase, the solution is dialyzed against a 0.1M pyrophosphate buffer solution (pH 8.5) for 2 days with three liquid-exchanges to remove potassium bromide to obtain apo-D-amino acid oxidase.

### Preparation Example 12

A PQQ-modified FAD is prepared through the process below. A 10 mM portion of PQQ is added to a 0.01 μM HEPES buffer solution (pH 7.3) containing 1 mM of N⁶-(2-aminoethyl) FAD described in Preparation Example 4 and 10 mM of 1-ethyl-3-(3-dimethylaminopropyl) carbodiimide. The mixture is stirred for 2 hours, and ultrafiltered to eliminate excessive PQQ to obtain PQQ-modified FAD.

### Preparation Example 13

A ferrocene-modified FAD is prepared in the same manner as in Preparation Example 12 except that the ferrocene derivative described in Preparation Example 5 is used instead of PQQ.

### Preparation Example 14

A PQQ-modified glucose oxidase is prepared through the process below. To a solution of 4 mg/mL of apoglucose oxidase prepared in Preparation Example 10 in a 0.1M phosphate buffer solution (pH 7.0), is added 0.2 mg/mL of the PQQ-modified FAD described in Preparation Example 12. The mixture is stirred at 25° C. for 4 hours and at 4° C. for 12 hours, and ultrafiltered to eliminate excessive PQQ to obtain PQQ-modified glucose oxidase.

### Preparation Example 15

A ferrocene-modified glucose oxidase is prepared in the same manner as in Preparation Example 14 except that the ferrocene derivative obtained in Preparation Example 13 is used instead of PQQ-modified FAD.

### Preparation Example 16

A ferrocene-modified D-amino acid oxidase is prepared in the same manner as in Preparation Example 15 except that the apo-amino acid oxidase described in Preparation Example 11 is used instead of apoglucose oxidase described in Preparation Example 10 described in Preparation Example 14 described in Preparation Example 15.

### Preparation Example 17

An enzyme electrode for cathode of a fuel cell is prepared as described below. In a sample tube, the osmium complex polymer described in Preparation Example 9 is dissolved in 5 mL of water at a concentration of 10 mg/mL. Thereto, are added 1 mL of 0.2M citrate buffer solution (pH 5) and 1 mL of an aqueous 30 mg/mL solution of laccase (Coriolus hirsutus). After stirring, thereto is added 2 mL of aqueous 10 mg/mL polyethylene glycol diglycidyl ether solution. The mixture is stirred. Separately, a sheet of a nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is cut in 1 cm square, washed, and subjected to UV-ozone treatment. This foamed nickel alloy sheet is immersed in the above-prepared enzyme-osmium polymer solution, then taken out and dried in a desiccator for 2 days to prepare an enzyme electrode.

The processes for preparation of enzyme electrodes of the present invention are described below.

### Example 1

In a sample tube, 1 mL of an aqueous solution containing 0.1 M sodium hydrogencarbonate and 40 mg/mL of the PQQ-modified glucose oxidase described in Preparation Example 14 is prepared. Thereto, is added 0.5 mL of an aqueous 7 mg/mL sodium periodate solution. The mixture is stirred in the dark for one hour. Thereto are added 6 mL of aqueous 10% polypyrrole solution and a mixture of 0.4 mL of an aqueous 2.5 mg/mL polyethylene glycol diglycidyl ether solution and 0.42 g of potassium hexacyanoferrate (III). Separately, a gold sheet (1 cm square, 0.3 mm thick, Nilaco) is cleaned and subjected to UV-ozone treatment. This gold sheet is immersed in the above-prepared mixture solution, then taken out and dried in a desiccator for 2 days to prepare an enzyme electrode.

### Example 2

An enzyme electrode is prepared in the same manner as in Example 1, except that a 10% polyaniline solution is used instead of the 10% polypyrrole solution, and 0.63 g of octacyanotungstate (IV) is used instead of 0.42 g of potassium hexacyanoferrate (III).

### Example 3

In a sample tube, 1 mL of an aqueous solution containing 0.1 M sodium hydrogencarbonate and 40 mg/mL of the PQQ-modified glucose oxidase described in Preparation Example 14 is prepared. Thereto, is added 0.5 mL of an aqueous 0.7 mg/mL sodium periodate solution. The mixture is stirred in the dark for one hour. Thereto are added 6 mL of aqueous 10 mg/mL solution of the osmium complex polymer described in Preparation Example 6 and 0.4 mL of an aqueous 2.5 mg/mL polyethylene glycol diglycidyl ether solution. Separately, a gold sheet (1 cm square, 0.3 mm thick, Nilaco) is cleaned and subjected to UV-ozone treatment. This gold sheet is immersed in the above-prepared mixture solution, then taken out and dried in a desiccator for 2 days to prepare an enzyme electrode.

### Example 4

In a sample tube, 1 mL of an aqueous solution containing 0.1 M sodium hydrogencarbonate and 40 mg/mL of the PQQ-modified glucose oxidase described in Preparation Example 14 is prepared. Thereto, is added 0.5 mL of an aqueous 7 mg/mL sodium periodate solution. The mixture is stirred in the dark for one hour. Thereto are added 6 mL of aqueous 4 mg/mL solution of polyvinylimidazole described in Preparation Example 6 and a mixture of 0.4 mL of an aqueous 2.5 mg/mL polyethylene glycol diglycidyl ether solution and 0.42 g of potassium hexacyanoferrate (III). Separately, a gold sheet (1 cm square, 0.3 mm thick, Nilaco) is cleaned and subjected to UV-ozone treatment. This gold sheet is immersed in the above-prepared mixture solution, then taken out and dried in a desiccator for 2 days to prepare an enzyme electrode.

### Example 5

An enzyme electrode is prepared in the same manner as in Example 4 except that 0.63 g of potassium octacyanotungstate (IV) is used in stead of 0.42 g of potassium hexacyanoferrate (III)

### Example 6

An enzyme electrode is prepared in the same manner as in Example 5 except that the ferrocene-modified glucose oxidase described in Preparation Example 15 is used instead of PQQ-modified glucose oxidase.

### Example 7

An enzyme electrode is prepared in the same manner as in Example 6 except that the ferrocene-modified amino acid oxidase described in Preparation Example 16 is used instead of ferrocene-modified glucose oxidase.

### Example 8

An enzyme electrode is prepared in the same manner as in Example 4 except that polyvinylpyridine (av Biol wt: 150,000) is used instead of the polyvinylimidazole described in Preparation Example 6.

### Example 9

An enzyme electrode is prepared in the same manner as in Example 5 except that poly-L-lysine (ay mol wt: 150,000) is used instead of the polyvinylimidazole described in Preparation Example 6.

### Example 10

An enzyme electrode is prepared in the same manner as in Example 3 except that ferrocene-containing polymer described in Preparation Example 7 is used instead of the osmium complex polymer described in preparation Example 6

### Example 11

An enzyme electrode is prepared in the same manner as in Example 1 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 12

An enzyme electrode is prepared in the same manner as in Example 2 except that foamed nickel alloy (Mitsubishi Materials Corp.; constituting elements: Ni, Cr, Ti, Nb, Al, Mn, Si, and C; 0.5 mm thick-; plated with 0.5 μm thick gold; pore size: 50 μm) is used instead of the gold sheet.

### Example 13

An enzyme-electrode is prepared in the same manner as in Example 3 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 14

An enzyme electrode is prepared in the same manner as in Example 4 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 15

An enzyme electrode is prepared in the same manner as in Example 5 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 16

An enzyme electrode is prepared in the same manner as in Example 6 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 17

An enzyme electrode is prepared in the same manner as in Example 7 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 18

An enzyme electrode is prepared in the same manner as in Example 8 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 19

An enzyme electrode is prepared in the same manner as in Example 9 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of, the gold sheet.

### Example 20

An enzyme electrode is prepared in the same manner as in Example 10 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Example 21

An enzyme electrode is prepared in the same manner as in Example 3 except that the conductive member constituted of poly(3,4-ethylenedioxythiphene) containing numerous voids described in Preparation Example 1 is used instead of the gold sheet.

### Example 22

An enzyme electrode is prepared in the same manner as in Example 3 except that the conductive member constituted of ITO described in Preparation Example 2 is used instead of the gold sheet.

### Example 23

An enzyme electrode is prepared in the same manner as in Example 3 except that a transparent conductive glass sheet constituted of fluorine-doped tin oxide (Asahi. Glass Co.; 8 Ω/sq) is used instead of the gold sheet.

### Example 24

An enzyme electrode is prepared in the same manner as in Example 3-except that the conductive member constituted of graphite having numerous voids described in Preparation Example 3 is used instead of the gold sheet.

### Comparative Example 1

A gold sheet (1 cm square; 0.3 mm thick; Nilaco) is cleaned and is subjected to UV-ozone treatment. This gold sheet is immersed in an aqueous 0.02M cystamine solution for 2 hours, then taken out, and washed with water to prepare a cystamine-modified conductive member. This conductive member is immersed in a 0.01M HEPES buffer solution containing 3 mM of PQQ and 10 mM of 1-ethyl-3-(3-dimethylaminopropyl) carbodiimide for one hour, and washed with water for PQQ modification of the conductive member. This PQQ-modified conductive member is immersed in a 0.01M HEPES buffer solution containing 1 mM N⁵ (2-aminoethyl) FAD described in Preparation Example 4 and 10 mM 1-ethyl-3-(3-dimethylaminopropyl) carbodiimide for 2 hours and washed with water for FAD modification of the conductive member. Further, this conductive member is immersed in a 0.1M phosphate buffer solution (pH 7.0) containing 4 mg/mL apoglucose oxidase described in Preparation Example 10 at 25° C. for 4 hours and at 4° C. for 12 hours, then taken out, and further immersed in a phosphate buffer solution for one hour to prepare an enzyme electrode.

### Comparative Example 2

An enzyme electrode is prepared in the same manner as in Example 1 except that the potassium hexacyanoferrate (III) is not added to the enzyme solution.

### Comparative Example 3

An enzyme electrode is prepared in the same manner as in Example 1 except that glucose oxidase (Aspergillus niger) is used instead of the PQQ-modified glucose oxidase described in Preparation Example 14;

### Comparative Example 4

An enzyme electrode is prepared in the same manner as in Comparative Example 2 except that a 10% polyaniline solution is used instead of the 10% polypyrrole solution.

### Comparative Example 5

An enzyme electrode is prepared in the same manner as in Comparative Example 3 except that a 10% polyaniline solution is used instead of the 10% polypyrrole solution, and 0.63 g of potassium octacyanotungstate (IV) is used instead of 0.42 g of the potassium hexacyanoferrate (III).

### Comparative Example 6

An enzyme electrode is prepared in the same manner as in Example 4 except that the potassium hexacyanoferrate (III) is not added to the enzyme solution.

### Comparative Example 7

An enzyme electrode is prepared in the same manner as in Example 3 except that glucose oxidase (Aspergillus niger) is used instead of the PQQ-modified glucose oxidase described in Preparation Example 14.

### Comparative Example 8

An enzyme electrode is prepared in the same manner as in Example 4 except that glucose oxidase (Aspergillus niger) is used instead of the PQQ-modified glucose oxidase described in Preparation Example 14.

### Comparative Example 9

An enzyme electrode is prepared in the same manner as in Example 5 except that glucose oxidase (Aspergillus niger) is used instead of the PQQ-modified glucose oxidase described in Preparation Example 14.

### Comparative Example 10

An enzyme electrode is prepared in the same manner as in Example 6 except that the potassium octacyanotungstate (IV) is not added to the enzyme solution.

### Comparative Example 11

An enzyme electrode is prepared in the same manner as in Example 7, except that the potassium octacyanotungstate (IV) is not added to the enzyme solution.

### Comparative Example 12

An enzyme electrode is prepared in the same manner as in Example 7 except that D-amino acid oxidase (swine kidney) is used instead of the ferrocene-modified amino acid oxidase.

### Comparative Example 13

An enzyme electrode is prepared in the same manner as in Example 3 except that the osmium complex polymer described in Preparation Example 8 is used instead of the osmium complex polymer described in Preparation Example 6.

### Comparative Example 14

An enzyme electrode is prepared in the same manner as in Example 4 except that the potassium hexacyanomanganate (III) is used instead of potassium hexacyanoferrate (III).

### Comparative Example 15

An enzyme electrode is prepared in the same manner as in Comparative Example 13 except that the ferrocene-modified glucose oxidase described in Preparation Example 15 is used instead of the PQQ-modified glucose oxidase described in Preparation Example 14.

### Comparative Example 16

An enzyme electrode is prepared in the same manner as in Example 6 except that the potassium hexacyanomanganate (III) is used instead of potassium octacyanotungstate (IV).

### Comparative Example 17

An enzyme electrode is prepared in the same manner as in Comparative Example 6 except that polyvinylpyridine (av mol wt: 150,000) is used instead of the polyvinylimidazole described in Preparation Example 6.

### Comparative Example 18

An enzyme electrode is prepared in the same manner as in Comparative Example 8 except that polyvinylpyridine (av mol wt: 150,000) is used instead of the polyvinylimidazole described in Preparation Example 6.

### Comparative Example 19

An enzyme electrode is prepared in the same manner as in Example 9 except that the potassium octacyanotungstate (IV) is not added to the enzyme solution.

### Comparative Example 20

An enzyme electrode is prepared in the same manner as in Example 9 except that glucose oxidase (Aspergillus niger) is used instead of the PQQ-modified glucose oxidase described in Preparation Example 14.

### Comparative Example 21

An enzyme electrode is prepared in the same manner as in Example 10 except that glucose oxidase (Aspergillus niger) is used instead of the PQQ-modified glucose oxidase described in Preparation Example 14.

### Comparative Example 22

An enzyme electrode is prepared in the same manner as in Comparative Example 2 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 23

An enzyme electrode is prepared in the same manner as in Comparative Example 3 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 24

An enzyme electrode is prepared in the same manner as in Comparative Example 4 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 25

An enzyme electrode is prepared in the same manner as in Comparative Example 5 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 26

An enzyme electrode is prepared in the same manner as in Comparative Example 6 except that foamed nickel alloy (Mitsubishi Materials Corp.; constituting elements: Ni, Cr, Ti, Nb, Al, Mn, Si, and C; 0.5 mm thick; plated with 0.5μm thick gold; pore size: 50 μm) is used instead of the gold sheet.

### Comparative Example 27

An enzyme electrode is prepared in the same manner as in Comparative Example 7 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 28

An enzyme electrode is prepared in the same manner as in Comparative Example 8 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 29

An enzyme electrode is prepared in the same manner as in Comparative Example 9 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 30

An enzyme electrode is prepared in the same manner as in Comparative Example 10 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 31

An enzyme electrode is prepared in the same manner as in Comparative Example 11 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 32

An enzyme electrode is prepared in the same manner as in Comparative Example 12 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 33

An enzyme electrode is prepared in the same manner as in Comparative Example 13 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 34

An enzyme electrode is prepared in the same manner as in Comparative Example 14 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 35

An enzyme electrode is prepared in the same manner as in Comparative Example 15 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 36

An enzyme electrode is prepared in the same manner as in Comparative Example 16 except that foamed nickel alloy (Mitsubishi Materials Corp.; constituting elements. Ni, Cr, Ti, Nb, Al, Mn, Si, and C; 0.5 mm thick; plated with 0.5μm thick gold; pore size: 50 μm) is used instead of the gold sheet.

### Comparative Example 37

An enzyme electrode is prepared in the same manner as in Comparative Example 17 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 38

An enzyme electrode is prepared in the same manner as in Comparative Example 18 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 39

An enzyme electrode is prepared in the same manner as in Comparative Example 19 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 40

An enzyme electrode is prepared in the same manner as in Comparative Example 20 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 41

An enzyme electrode is prepared in the same manner as in Comparative Example 21 except that foamed nickel alloy (Mitsubishi Materials Corp.; constituting elements: Ni, Cr, Ti, Nb, Al, Mn, Si, and C; 0.5 mm thick; plated with 0.5 μm thick gold; pore size: 50 μm) is used instead of the gold sheet.

### Comparative Example 42

An enzyme electrode is prepared in the same manner as in Example 6 except that the conductive member constituted of poly(3,4-ethylenedioxythiphene having numerous voids described in Preparation Example 1 is used instead of the gold sheet.

### Comparative Example 43

An enzyme electrode is prepared in the same manner as in Example 7 except that the conductive member constituted of poly(3,4-ethylenedioxythiphene) having numerous voids described in Preparation Example 1 is used instead of the gold sheet.

### Comparative Example 44

An enzyme electrode is prepared in the same manner as in Example 6 except that the conductive member constituted of ITO described in Preparation Example 2 is used instead of the gold sheet.

### Comparative Example 45

An enzyme electrode is prepared in the same manner as in Example 7 except that the conductive member constituted of ITO described in Preparation Example 2 is used instead of the gold sheet.

### Comparative Example 46

An enzyme electrode is prepared in the same manner as in Example 6 except that a transparent conductive glass sheet constituted of fluorine-doped tin oxide (Asahi Glass Co./8 Ω/sq) is used instead of the gold sheet.

### Comparative Example 47

An enzyme electrode is prepared in the same manner as in Example 7 except that a transparent conductive glass sheet constituted of fluorine-doped tin oxide (Asahi Glass Co.; 8 Ω/sq) is used instead of the gold sheet.

### Comparative Example 48

An enzyme electrode is prepared in the same manner as in Example 6 except that the conductive member constituted of graphite having numerous voids described in Preparation Example 3 is used instead of the gold sheet.

### Comparative Example 49

An enzyme electrode is prepared in the same manner as in Example 7 except that the conductive member constituted of graphite having numerous voids described in Preparation Example 3 is used instead of the gold sheet.

### Example 25

Sensors are prepared with enzyme electrodes described in Examples 1-24 and Comparative Examples 1-49. A three-electrode cell (FIG. 6) is used which has an enzyme electrode as the working electrode with a leading wire, an Ag/AgCl electrode as the reference electrode, and a platinum wire as the counter electrode. The electrolyte for a glucose oxidase-employing enzyme electrode is a 20 mM phosphate buffer solution (pH 7.2) containing 15 mM glucose and 0.1M NaCl. The electrolyte for a D-amino acid oxidase-employing enzyme electrode is a 0.1M pyrophosphate buffer solution (pH 8.5) containing 15 mM D-alanine. In the measurement, the respective electrodes are connected to a potentiostat (Toho Giken K. K.; Model 2000), and a steady-state-electric current is recorded by application of a potential of 0.5 V (vs Ag/AgCl) in a nitrogen atmosphere. A water-jecketed cell is used as the vessel. The measurement temperature is kept at 37° C. by a constant temperature bath. Tables 1(1)-1(3) show the results.

TABLE 1(1)

| | Current density | Current density |
|---|---|---|
| Enzyme | (No substrate) | (Substrate added) |
| electrode | μA/cm² | μA/cm² |
| Example 1 | 4 | 560 |
| Example 2 | 7 | 520 |
| Example 3 | 7 | 950 |
| Example 4 | 7 | 720 |
| Example 5 | 4 | 690 |
| Example 6 | 7 | 630 |
| Example 7 | 2 | 150 |
| Example 8 | 1 | 540 |
| Example 9 | 8 | 540 |
| Example 10 | 9 | 460 |
| Example 11 | 51 | 4100 |
| Example 12 | 56 | 3500 |
| Example 13 | 61 | 7500 |
| Example 14 | 59 | 5200 |
| Example 15 | 56 | 5500 |
| Example 16 | 42 | 4600 |
| Example 17 | 13 | 1100 |
| Example 18 | 11 | 3600 |
| Example 19 | 28 | 4000 |
| Example 20 | 69 | 3700 |
| Example 21 | 15 | 4800 |
| Example 22 | 26 | 2800 |
| Example 23 | 16 | 1500 |
| Example 24 | 15 | 1100 |

TABLE 1(2)

| | Current density | Current density |
|---|---|---|
| Enzyme | (No substrate) | (Substrate added) |
| electrode | μA/cm² | μA/cm² |
| Comp. Ex. 1 | 1 | 150 |
| Comp. Ex. 2 | 2 | 160 |
| Comp. Ex. 3 | 1 | 98 |
| Comp. Ex. 4 | 1 | 140 |
| Comp. Ex. 5 | 0 | 170 |
| Comp. Ex. 6 | 1 | 46 |
| Comp. Ex. 7 | 1 | 350 |
| Comp. Ex. 8 | 2 | 280 |
| Comp. Ex. 9 | 6 | 320 |
| Comp. Ex. 10 | 0 | 28 |
| Comp. Ex. 11 | 0 | 5 |
| Comp. Ex. 12 | 1 | 95 |
| Comp. Ex. 13 | 0 | 0 |
| Comp. Ex. 14 | 0 | 0 |
| Comp. Ex. 15 | 0 | 0 |
| Comp. Ex. 16 | 0 | 0 |
| Comp. Ex. 17 | 1 | 49 |
| Comp. Ex. 18 | 1 | 180 |
| Comp. Ex. 19 | 1 | 39 |
| Comp. Ex. 20 | 4 | 230 |
| Comp. Ex. 21 | 3 | 190 |
| Comp. Ex. 22 | 5 | 1100 |
| Comp. Ex. 23 | 8 | 1400 |
| Comp. Ex. 24 | 5 | 950 |
| Comp. Ex. 25 | 18 | 1200 |

Comp. Ex.: Comparative Example

TABLE 1(3]

| | Current density | Current density |
|---|---|---|
| Enzyme | (No substrate) | (Substrate added) |
| electrode | μA/cm² | μA/cm² |
| Comp. Ex. 26 | 3 | 360 |
| Comp. Ex. 27 | 36 | 2400 |
| Comp. Ex. 28 | 5 | 1900 |
| Comp. Ex. 29 | 3 | 2100 |
| Comp. Ex. 30 | 3 | 220 |
| Comp. Ex. 31 | 0 | 37 |
| Comp. Ex. 32 | 10 | 740 |
| Comp. Ex. 33 | 0 | 0 |
| Comp. Ex. 34 | 0 | 0 |
| Comp. Ex. 35 | 0 | 0 |
| Comp. Ex. 36 | 0 | 0 |
| Comp. Ex. 37 | 4 | 360 |
| Comp. Ex. 38 | 14 | 1400 |
| Comp. Ex. 39 | 2 | 280 |
| Comp. Ex. 40 | 36 | 1800 |
| Comp. Ex. 41 | 21 | 1400 |
| Comp. Ex. 42 | 2 | 240 |
| Comp. Ex. 43 | 18 | 1700 |
| Comp. Ex. 44 | 1 | 140 |
| Comp. Ex. 45 | 12 | 1100 |
| Comp. Ex. 46 | 1 | 72 |
| Comp. Ex. 47 | 5 | 530 |
| Comp. Ex. 48 | 0 | 56 |
| Comp. Ex. 49 | 8 | 430 |

Comp. Ex.: Comparative Example

The sensors which employ an enzyme electrode of Examples 1-24 containing a first mediator for transferring electrons to or from the enzyme and a second mediator for transferring electrons to or from the first mediator, give a higher electric current density than corresponding enzyme electrodes prepared using only one kind of mediator. Presumably, introduction of the first mediator increases the enzyme/mediator electron transfer rate, and the entrapping immobilization increases the density of the immobilized enzyme in the conductive member. On the other hand, the second mediators used in the enzyme electrodes of Comparative Examples 13-16 have redox potentials (reduction-oxidation potentials) of −0.19 V (vs Ag/AgCl, Examples 13, 15) and −1.02 V (vs Ag/AgCl, Examples 14, 16) according to literature. These redox potentials, (reduction-oxidation potentials) are more negative than that of the corresponding first mediators (−0.14 V vs Ag/AgCl at the most negative). Therefore, in these Comparative Examples, electron transfer does not occur between the first mediator and the second mediator, resulting in a low current density. Thus, the enzyme electrode having the first mediator for transfer of electrons to or from the enzyme and the second mediator for transfer of electrons to or from the first mediator can improve the sensitivity of the sensor.

### Example 26

Fuel cells are prepared with enzyme electrodes described in Examples 1-24 and Comparative Examples 1-49. The cell has a two-electrode constitution (FIG. 7) which has an enzyme electrode with a leading wire as the working electrode, an enzyme electrode described in Preparation Example 17 as the counter electrode, and a porous polypropylene film (20 μm thick) held between the electrodes. The electrolyte is a 0.2M citrate buffer solution (pH 5.0). The anode fuel is 15 mM glucose for glucose oxidase-employing electrodes, and 15 mM D-alanine for D-amino acid oxidase-employing electrodes: the cathode fuel is saturated oxygen. In the measurement, with the respective electrodes connected to a potentiostat (Toho Giken K. K.; Model 2000), the voltage-current characteristics are derived by changing the voltage from −1.2 V to 0.1 V at a rate of 1 mV/sec. The measurement vessel is a water-jacketed cell, and the measurement temperature is kept at 37° C. by a constant temperature bath. Tables 2(1)-2 (3) show the results.

TABLE 2(1)

| | | Short-circuit | Maximum |
|---|---|---|---|
| | | current density | output |
| Anode | Cathode | μA/cm² | μW/cm² |
| Example 1 | Prepn. Ex. 17 | 450 | 53 |
| Example 2 | Prepn. Ex. 17 | 420 | 17 |
| Example 3 | Prepn. Ex. 17 | 760 | 231 |
| Example 4 | Prepn. Ex. 17 | 580 | 76 |
| Example 5 | Prepn. Ex. 17 | 550 | 22 |
| Example 6 | Prepn. Ex. 17 | 500 | 20 |
| Example 7 | Prepn. Ex. 17 | 120 | 5 |
| Example 8 | Prepn. Ex. 17 | 430 | 56 |
| Example 9 | Prepn. Ex. 17 | 430 | 18 |
| Example 10 | Prepn. Ex. 17 | 370 | 78 |
| Example 11 | Prepn. Ex. 17 | 3000 | 317 |
| Example 12 | Prepn. Ex. 17 | 2800 | 100 |
| Example 13 | Prepn. Ex. 17 | 3000 | 818 |
| Example 14 | Prepn. Ex. 17 | 3000 | 320 |
| Example 15 | Prepn. Ex. 17 | 3000 | 111 |
| Example 16 | Prepn. Ex. 17 | 3000 | 112 |
| Example 17 | Prepn. Ex. 17 | 870 | 32 |
| Example 18 | Prepn. Ex. 17 | 2900 | 324 |
| Example 19 | Prepn. Ex. 17 | 3000 | 112 |
| Example 20 | Prepn. Ex. 17 | 2900 | 555 |
| Example 21 | Prepn. Ex. 17 | 3000 | 403 |
| Example 22 | Prepn. Ex. 17 | 2300 | 557 |
| Example 23 | Prepn. Ex. 17 | 1200 | 237 |
| Example 24 | Prepn. Ex. 17 | 870 | 49 |

Prepn. Ex.: Preparation Example

TABLE 2(2)

| | | Short-circuit | Maximum |
|---|---|---|---|
| | | current density | output |
| Anode | Cathode | μA/cm² | μW/cm² |
| Comp. Ex. 1 | Prepn. Ex. 17 | 120 | 54 |
| Comp. Ex. 2 | Prepn. Ex. 17 | 130 | 57 |
| Comp. Ex. 3 | Prepn. Ex. 17 | 79 | 10 |
| Comp. Ex. 4 | Prepn. Ex. 17 | 110 | 49 |
| Comp. Ex. 5 | Prepn. Ex. 17 | 140 | 6 |
| Comp. Ex. 6 | Prepn. Ex. 17 | 37 | 16 |
| Comp. Ex. 7 | Prepn. Ex. 17 | 280 | 84 |
| Comp. Ex. 8 | Prepn. Ex. 17 | 220 | 26 |
| Comp. Ex. 9 | Prepn. Ex. 17 | 260 | 11 |
| Comp. Ex. 10 | Prepn. Ex. 17 | 22 | 2 |
| Comp. Ex. 11 | Prepn. Ex. 17 | 4 | 0 |
| Comp. Ex. 12 | Prepn. Ex. 17 | 76 | 3 |
| Comp. Ex. 13 | Prepn. Ex. 17 | 0 | 0 |
| Comp. Ex. 14 | Prepn. Ex. 17 | 0 | 0 |
| Comp. Ex. 15 | Prepn. Ex. 17 | 0 | 0 |
| Comp. Ex. 16 | Prepn. Ex. 17 | 0 | 0 |
| Comp. Ex. 17 | Prepn. Ex. 17 | 39 | 18 |
| Comp. Ex. 18 | Prepn. Ex. 17 | 150 | 18 |
| Comp. Ex. 19 | Prepn. Ex. 17 | 31 | 14 |
| Comp. Ex. 20 | Prepn. Ex. 17 | 180 | 8 |
| Comp. Ex. 21 | Prepn. Ex. 17 | 150 | 32 |
| Comp. Ex. 22 | Prepn. Ex. 17 | 890 | 354 |
| Comp. Ex. 23 | Prepn. Ex. 17 | 1100 | 120 |
| Comp. Ex. 24 | Prepn. Ex. 17 | 760 | 315 |
| Comp. Ex. 25 | Prepn. Ex. 17 | 960 | 36 |

Comp. Ex.: Comparative Example
Prepn. Ex.: Preparation Example

TABLE 2(3)

| | | Short-circuit | Maximum |
|---|---|---|---|
| | | current density | output |
| Anode | Cathode | μA/cm² | μW/cm² |
| Comp. Ex. 26 | Prepn. Ex. 17 | 290 | 119 |
| Comp. Ex. 27 | Prepn. Ex. 17 | 1900 | 523 |
| Comp. Ex. 28 | Prepn. Ex. 17 | 1500 | 164 |
| Comp. Ex. 29 | Prepn. Ex. 17 | 1700 | 60 |
| Comp. Ex. 30 | Prepn. Ex. 17 | 180 | 12 |
| Comp. Ex. 31 | Prepn. Ex. 17 | 29 | 2 |
| Comp. Ex. 32 | Prepn. Ex. 17 | 590 | 22 |
| Comp. Ex. 33 | Prepn. Ex. 17 | 0 | 0 |
| Comp. Ex. 34 | Prepn. Ex. 17 | 0 | 0 |
| Comp. Ex. 35 | Prepn. Ex. 17 | 0 | 0 |
| Comp. Ex. 36 | Prepn. Ex. 17 | 0 | 0 |
| Comp. Ex. 37 | Prepn. Ex. 17 | 290 | 114 |
| Comp. Ex. 38 | Prepn. Ex. 17 | 1200 | 132 |
| Comp. Ex. 39 | Prepn. Ex. 17 | 230 | 96 |
| Comp. Ex. 40 | Prepn. Ex. 17 | 1400 | 56 |
| Comp. Ex. 41 | Prepn. Ex. 17 | 1100 | 194 |
| Comp. Ex. 42 | Prepn. Ex. 17 | 190 | 41 |
| Comp. Ex. 43 | Prepn. Ex. 17 | 1400 | 190 |
| Comp. Ex. 44 | Prepn. Ex. 17 | 120 | 39 |
| Comp. Ex. 45 | Prepn. Ex. 17 | 850 | 208 |
| Comp. Ex. 46 | Prepn. Ex. 17 | 58 | 17 |
| Comp. Ex. 47 | Prepn. Ex. 17 | 430 | 87 |
| Comp. Ex. 48 | Prepn. Ex. 17 | 45 | 4 |
| Comp. Ex. 49 | Prepn. Ex. 17 | 340 | 18 |

Comp. Ex.: Comparative Example
Prepn. Ex.: Preparation Example

The fuel cells, which employ an enzyme electrode of Examples 1-24 containing a first mediator for transferring electrons to or from the enzyme and a second mediator for transferring electrons to or from the first mediator, give a higher short-circuit current density than corresponding enzyme electrodes prepared using only one kind of mediator. Presumably, introduction of the first mediator increases the enzyme/mediator electron transfer rate, and the entrapping immobilization increases the density of the immobilized enzyme in the conductive member. On the other hand, the second mediators used in the enzyme electrodes of Comparative Examples 13-16 have redox potentials (reduction-oxidation potentials) of −0.19 V (vs Ag/AgCl, Comparative Examples 13, 15) and −1.02 V (vs Ag/AgCl, Comparative Examples 14, 16) according to literature. These redox potentials (reduction-oxidation potentials) are more negative than that of the corresponding first mediators (−0.14 V vs Ag/AgCL at the most negative). Therefore, in these Comparative Examples, electron transfer does not occur between the first mediator and the second mediator, resulting in a low current density. In some of the fuel cells of Examples, the maximum output is lower than that of the corresponding fuel cells of Comparative Examples without the mediators owing to a voltage loss by introduction of plural mediators. However, as a whole, the fuel cells employing the enzyme electrodes of Examples give higher output than the corresponding to the fuel cells of Comparative Examples without the mediators. Thus, the enzyme electrode having the first mediator for transfer of electrons to or from the enzyme and the second mediator for transfer of electrons to or from the first mediator can improve the output of the fuel cells.

### Example 27

Electrochemical reactors are prepared with enzyme electrodes described in Examples 1-24 and Comparative Examples 1-49. A three-electrode cell (FIG. 6) is used which has an enzyme electrode as the working electrode, an Ag/AgCl electrode as the reference electrode, and a platinum wire as the counter electrode. The electrolyte solution is a 20 mM phosphate buffer solution (pH 7.2) containing 0.1M NaCl, 10 mM glucose, and 10 mM D-alanine. In the measurement, a potential of 0.4 V vs Ag/AgCl is applied for 100 minutes in a water-jecketed cell in a nitrogen atmosphere. The formed product is quantitatively determined by high-speed liquid chromatography. Tables 3(1)-3 (3) show the results.

TABLE 3(1)

| | Reaction | | Detected |
|---|---|---|---|
| Enzyme | charge | Detected | quantity |
| electrode | mC | substance | μmol |
| Example 1 | 3200 | Gluconolactone | 32 |
| Example 2 | 2900 | Gluconolactone | 30 |
| Example 3 | 5400 | Gluconolactone | 56 |
| Example 4 | 4000 | Gluconolactone | 41 |
| Example 5 | 4100 | Gluconolactone | 41 |
| Example 6 | 3400 | Gluconolactone | 37 |
| Example 7 | 800 | Pyruvic acid | 8 |
| Example 8 | 2900 | Gluconolactone | 32 |
| Example 9 | 3200 | Gluconolactone | 34 |
| Example 10 | 2600 | Gluconolactone | 27 |
| Example 11 | 25000 | Gluconolactone | 250 |
| Example 12 | 21000 | Gluconolactone | 210 |
| Example 13 | 44000 | Gluconolactone | 450 |
| Example 14 | 29000 | Gluconolactone | 310 |
| Example 15 | 30000 | Gluconolactone | 300 |
| Example 16 | 26000 | Gluconolactone | 280 |
| Example 17 | 6300 | Pyruvic acid | 64 |
| Example 18 | 20000 | Gluconolactone | 200 |
| Example 19 | 22000 | Gluconolactone | 230 |
| Example 20 | 20000 | Gluconolactone | 210 |
| Example 21 | 28000 | Gluconolactone | 290 |
| Example 22 | 15000 | Gluconolactone | 170 |
| Example 23 | 7900 | Gluconolactone | 86 |
| Example 24 | 6300 | Gluconolactone | 65 |

TABLE 3(2)

| | Reaction | | Detected |
|---|---|---|---|
| Enzyme | charge | Detected | quantity |
| electrode | mC | substance | μmol |
| Comp. Ex. 1 | 830 | Gluconolactone | 9 |
| Comp. Ex. 2 | 880 | Gluconolactone | 9 |
| Comp. Ex. 3 | 550 | Gluconolactone | 6 |
| Comp. Ex. 4 | 770 | Gluconolactone | 8 |
| Comp. Ex. 5 | 940 | Gluconolactone | 10 |
| Comp. Ex. 6 | 260 | Gluconolactone | 3 |
| Comp. Ex. 7 | 2000 | Gluconolactone | 21 |
| Comp. Ex. 8 | 1600 | Gluconolactone | 16 |
| Comp. Ex. 9 | 1900 | Gluconolactone | 21 |
| Comp. Ex. 10 | 160 | Gluconolactone | 2 |
| Comp. Ex. 11 | 26 | Pyruvic acid | 0 |
| Comp. Ex. 12 | 540 | Pyruvic acid | 6 |
| Comp. Ex. 13 | 0 | Gluconolactone | 0 |
| Comp. Ex. 14 | 0 | Gluconolactone | 0 |
| Comp. Ex. 15 | 0 | Gluconolactone | 0 |
| Comp. Ex. 16 | 0 | Gluconolactone | 0 |
| Comp. Ex. 17 | 270 | Gluconolactone | 3 |
| Comp. Ex. 18 | 1000 | Gluconolactone | 11 |
| Comp. Ex. 19 | 210 | Gluconolactone | 2 |
| Comp. Ex. 20 | 1300 | Gluconolactone | 13 |
| Comp. Ex. 21 | 1100 | Gluconolactone | 12 |
| Comp. Ex. 22 | 6700 | Gluconolactone | 71 |
| Comp. Ex. 23 | 8100 | Gluconolactone | 88 |
| Comp. Ex. 24 | 5200 | Gluconolactone | 54 |
| Comp. Ex. 25 | 7100 | Gluconolactone | 77 |

Comp. Ex.: Comparative Example

TABLE 3(3)

| | Reaction | | Detected |
|---|---|---|---|
| Enzyme | charge | Detected | quantity |
| electrode | mC | substance | μmol |
| Comp. Ex. 26 | 2000 | Gluconolactone | 22 |
| Comp. Ex. 27 | 14000 | Gluconolactone | 150 |
| Comp. Ex. 28 | 11000 | Gluconolactone | 110 |
| Comp. Ex. 29 | 12000 | Gluconolactone | 120 |
| Comp. Ex. 30 | 1300 | Gluconolactone | 13 |
| Comp. Ex. 31 | 220 | Pyruvic acid | 2 |
| Comp. Ex. 32 | 4000 | Pyruvic acid | 41 |
| Comp. Ex. 33 | 0 | Gluconolactone | 0 |
| Comp. Ex. 34 | 0 | Gluconolactone | 0 |
| Comp. Ex. 35 | 0 | Gluconolactone | 0 |
| Comp. Ex. 36 | 0 | Gluconolactone | 0 |
| Comp. Ex. 37 | 2100 | Gluconolactone | 22 |
| Comp. Ex. 38 | 8100 | Gluconolactone | 84 |
| Comp. Ex. 39 | 1600 | Gluconolactone | 16 |
| Comp. Ex. 40 | 10000 | Gluconolactone | 110 |
| Comp. Ex. 41 | 8000 | Gluconolactone | 86 |
| Comp. Ex. 42 | 1500 | Gluconolactone | 15 |
| Comp. Ex. 43 | 9900 | Gluconolactone | 98 |
| Comp. Ex. 44 | 850 | Gluconolactone | 9 |
| Comp. Ex. 45 | 6000 | Gluconolactone | 64 |
| Comp. Ex. 46 | 410 | Gluconolactone | 4 |
| Comp. Ex. 47 | 3100 | Gluconolactone | 32 |
| Comp. Ex. 48 | 310 | Gluconolactone | 3 |
| Comp. Ex. 49 | 2500 | Gluconolactone | 27 |

Comp. Ex.: Comparative Example

After the reaction, except the reactor employing the enzyme electrode of Comparative Examples 13-16, from the electrolytic solution of the electrochemical reactor having an enzyme electrode having a glucose-specific enzyme, gluconolactone is detected without detecting pyruvic acid, whereas from the electrolytic solution of the electrochemical reactor having an enzyme electrode having a D-alanine-specific enzyme, pyruvic acid is detected without detecting gluconolactone. Thus in any of the enzyme electrode-employing reactor, the reaction proceeds by selection of the substrate. On the other hand, the second mediators used in the enzyme electrodes of Comparative Examples 13-16 have redox potentials (reduction-oxidation potentials) of −0.19 V (vs Ag/AgCl, Comparative Examples 13, 15) and −1.02 V (vs Ag/AgCl, Comparative Examples 14, 16) according to literature. These redox potentials (reduction-oxidation potentials) are more negative than that of the corresponding first mediators (−0.14 V vs Ag/AgCl at the most negative). Therefore, in these Comparative Examples electron transfer does not occur between the first mediator and the second mediator, resulting in a low current density. Thus, the enzyme electrode having the first mediator for transfer of electrons to or from the enzyme and the second mediator for transfer of electrons to or from the first mediator can increase the reaction rate and increase of the sensitivity of the electrochemical reaction.

This application claims priority from Japanese Patent Application No. 2004-216377 filed on Jul. 23, 2004, which is hereby incorporated by reference herein.

1. An enzyme electrode comprising a conductive member and an enzyme, wherein a first mediator and a second mediator are immobilized by a carrier onto the conductive member, the first mediator and the second mediator having different redox potentials.

2. The enzyme electrode according to claim 1 wherein the first mediator has a redox potential more negative than the redox potential of the second mediator, and the electron transfer reaction rate between the second mediator and the conductive member is higher than the electron transfer reaction rate between the first mediator and the conductive member.

3. The enzyme electrode according to claim 1, wherein the first mediator has a redox potential more positive than the redox potential of the second mediator, and the electron transfer reaction rate between the second mediator and the conductive member is higher than the electron transfer reaction rate between the first mediator and the conductive member.

4. The enzyme electrode according to claim 1, wherein the first mediator serves to transfer electrons to or from the enzyme, and the second mediator serves to transfer electrons to or from the first mediator.

5. The enzyme electrode according to claim 1, wherein the conductive member has a porous structure.

6. The enzyme electrode according to claim 1, wherein the first mediator and the second mediator are respectively at least one substance selected from metal complexes, quinones, heterocyclic compounds, nicotinamide derivatives, and flavin derivatives.

7. The enzyme electrode according to claim 1, wherein the first mediator has a redox potential more negative than the redox potential of the second mediator, and is employed as an anode.

8. The enzyme electrode according to claim 1, wherein the second mediator has a redox potential more positive than the redox potential of the second mediator, and is employed as a cathode.

9. A sensor, employing the enzyme electrode set forth in any of claims 1 to 8 as a detection portion for detecting a substance.

10. A fuel cell, employing the enzyme electrode set forth in any of claims 1 to 8 as at least one of anode and a cathode.

11. An electrochemical reactor, employing the enzyme electrode set forth in any of claims 1 to 5 as a reaction electrode.
